(19) United States
(12) Patent Application Publication
(10) Pub. No.: US 2023/0340449 A1
(43) Pub. Date: Oct. 26, 2023

(54) THERMOSTABLE LIGASE WITH REDUCED SEQUENCE BIAS

(71) Applicant: QIAGEN BEVERLY LLC, Beverly, MA (US)

(72) Inventors: Ryan Charles Heller, Amesbury, MA (US); Thomas William Schoenfeld, Topsfield, MA (US); Patrick Barchard, Beverly, MS (US)

(21) Appl. No.: 18/016,584

(22) PCT Filed: Jun. 17, 2021
(86) PCT No.: PCT/US2021/037945

§ 371 Date: Jan. 17, 2023

Related U.S. Application Data
(60) Provisional application No. 63/053,258, filed on Jul. 17, 2020.

(30) Foreign Application Priority Data
Oct. 14, 2020 (EP) 20201876

(51) Int. Cl. C12N 9/00

(52) U.S. Cl. CPC C12N 9/93

(57) ABSTRACT

The present invention relates to a DNA and/or RNA ligase enzyme, its amino acid sequence, its nucleic acid sequence and to DNA and/or RNA ligase proteins encoded by these nucleic acid sequences, as well as nucleic acid or amino acid constructs comprising portions of the nucleic acid or amino acid sequence of the DNA and/or RNA ligase enzyme. The invention further relates to methods of using the DNA and/or RNA ligase enzyme for molecular biological assays and molecular diagnostic applications as well as to a kit containing the DNA and/or RNA ligase enzyme.

### FIELD OF THE INVENTION

The present invention is in the field of molecular biology, in particular in the field of enzymes and more particular in the field of ligases. It is also in the field of single-stranded nucleic acid circularization.

### BACKGROUND OF THE INVENTION

The invention relates to ligase enzymes, in particular thermostable ligases that are capable of template-independent intermolecular and/or intramolecular nucleic acid molecule ligation. Also included in the present invention are methods of using the thermostable ligase, in particular in single-stranded nucleic acid molecule circularization.

Enzymes, such as polymerases and ligases, are the workhorses in modern molecular biology and molecular diagnostics. Due to the dramatic improvements that were achieved in the fields of molecular biology and in molecular diagnostics, e.g. through the development and improvements in next generation sequencing (NGS), polymerase chain reaction (PCR), rolling circle amplification (RCA), and digital PCR (dPCR), highly efficient enzymes are greatly needed to further improve the current methods and assays and for the development of new methods in these technical fields.

Ligases are enzymes that can catalyze the joining of two molecules, e.g. nucleic acid molecules. Ribonucleic acid (RNA) and/or deoxyribonucleic acid (DNA) ligases are abundant in bacteriophage T4 infected cells and catalyze the ligation of a 5′-phosphoryl-terminated nucleic acid donor (RNA or DNA) to a 3′-hydroxyl-terminated nucleic acid acceptor (Silber et al., 1972. PNAS, Vol. 69, Nr. 10, doi: 10.1073/pnas.69.10.3009).

The RNA ligase 1 (Rnl1) family of enzymes, of which T4 Rnl1 is the founding member, is a class of enzymes responsible for the repair of programmed breaks in tRNA in vivo, countering a host defense mechanism. T4 Rnl1 has the ability to ligate single-stranded nucleic acids in vitro by catalyzing the formation of a phosphodiester bond between 5′-phosphate and 3′-hydroxyl ends of either single-stranded RNA or DNA (Omari et al., 2006. The Journal of Biological Chemistry, Vol. 283, Nr. 3, doi: 10.1074/jbc.M509658200). This includes both the intermolecular ligation of two different single-stranded DNA and/or RNA molecules or intramolecular ligation (circularization) of a single nucleic acid molecule without the requirement for a bridging or splint nucleic acid molecule. T4 Rnl1 has been essential for many molecular biology methods including, but not limited to, the 3′ end labeling of RNA, oligonucleotide synthesis, cDNA adapter ligation, rapid amplification of cDNA ends (RLM-RACE), ligation of single-stranded primer products for PCR (e.g. Kaluz et al., 1995. Biotechniques, Vol. 19, Nr. 2, 186; Tessier et al., 1986. Analytical Biochemistry, Vol. 158, Nr. 1, doi: 10.1016/0003-2697(86)90606-8; Middleton et al., 1985. Analytical Biochemistry, Vol. 144, nr. 1, doi: 10.1016/0003-2697(85)90091-0; Heckler et al., 1984. Biochemistry, Vol. 23, Nr. 7, doi: 10.1021/bi00302a020; Brennan et al., 1983. Methods in Enzymology, Vol. 100, doi: 10.1016/0076-6879(83)00044-0; Edwards et al., 1991. Nucleic Acids Research, Vol. 19, Nr. 19, doi: 10.1093/nar/19.19.5227; Liu & Gorovsky, 1993. Nucleic Acids research, Vol. 21, Nr. 21, doi: 10.1093/nar/21.21.4954). While able to circularize DNA and RNA, reactions with T4 Rnl1 are not efficient and often require a molecular crowding reagent and long incubation times (Harrison & Zimmerman, 1984. Nucleic Acids Research, Vol. 12, Nr 21, doi: 10.1093/nar/12.21.8235). In addition, because T4 Rnl1 is a mesophilic enzyme, reactions must be performed at low temperatures (Silber et al., 1972. PNAS, Vol. 69, Nr. 10, doi: 10.1073/pnas.69.10.3009) at which template single-stranded nucleic acids can form unwanted secondary structures that adversely affect reaction efficiency.

Some examples of thermophilic Rnl1 enzymes that have been characterized previously include the RM378 Rnl1 ligase from a thermophilic bacteriophage that infects the eubacterium Rhodothermus marinus and the TS2126 Rnl1 ligase that infects the thermophilic eubacterium Thermus scotoductus (Blondal et al., 2003. Nucleic Acids Research, Vol 31, No. 24, doi: 10.1093/nar/gkg914; Blondal et al., 2005. Nucleic Acid Research, Vol. 33, No. 1, doi:10.1093/nar/gki149). Both enzymes are moderately thermostable, with a temperature optimum approximately in the range of 60-70° C., conditions that would be expected to relax single-stranded template secondary structure. The TS2126 Rnl1 ligase showed higher levels of single-stranded ligation efficiency, favoring intramolecular circularization reactions. The enzyme was commercialized by Epicentre as CircLigase ssDNA Ligase, which catalyzes single-stranded circularization in an ATP-dependent manner with a low rate of end-to-end linear or circular concatemer formation. Subsequently, the TS2126 Rnl1 ligase was purified from cells in a manner that allowed for the predominately adenylated form to be isolated. This allowed for improved activity and increased circularization efficiency in reactions that are performed in an ATP-independent manner. The predominately adenylated form of the enzyme is commercially available from Epicentre as CircLigase II. The thermostable template-independent ligation activity of the TS2126 Rnl1 ligase has been utilized for the production of single-stranded circular templates for rolling-circle amplification (e.g. Gyanchandani et al., 2018. Scientific Reports, Vol. 8, Nr. 1, doi: 10.1038/s41598-018-35470-9) and rolling-circle transcription, isothermal nucleic acid amplification methods (Murakami et al., 2008. Nucleic Acids Research, Vol. 37, Nr. 3, doi: 10.1093/nar/gkn1014), amplification of low copy fragmented DNA for forensic applications (Tate et al., 2012. FSI Genetics, Vol. 6, Nr. 2, doi: 10.1016/j.fsigen.2011.04.011), and for several sequencing library preparation workflows (e.g. Lamm et al., 2011. Genome Research, Vol.21, doi: 10.1101/gr.108845.110; Lou et al., 2013. PNAS, Vol. 110, Nr. 49, doi: 10.1073/pnas.1319590110; Heyer et al., 2015. Nucleic Acids Research, Vol. 43, No. 1, doi: 10.1093/nar/gku1235), including whole-genome bisulfite sequencing (Miura et al., 2019. Nucleic Acids Research, Vol. 47, Nr. 15, Doi: 10.1093/nar/gkz435). Polidoros et al. (2006. BioTechniques Vol 41, Nr. 1, doi: 10.2144/000112205) described use of the template-independent TS2126 Rnl1 ligase as a step in a method for amplifying cDNA ends for random amplification of cDNA ends (RACE).

US20040058330A1 describes methods of using RM378 Rnl1 ligase or TS2126 Rnl1 ligase e.g. for the ligation of nucleotides or nucleic acids, the synthesis of an oligonucleotide polymer or a recombinant gene product, the ligation of probes to nucleic acids, the amplification of nucleic acids, ligation of 3′ label to mRNA, the formation of a nucleic acid library and in sequencing reactions of oligonucleotides.

US20040259123A1 discloses a heat-resistant DNA ligase obtained by cloning DNA ligase genes from the primitive extreme thermophile Aeropyrum pernix K1 strain. The activity of said ligase is not decreased by heat treatment at 100° C. for 1 hour.

US20090061481A1 describes a DNA ligase showing high thermal resistance and high DNA binding ability. Said heat resistant DNA ligase is derived from thermophilic bacteria such as Bacillus Stearothermophilus, hyperthermophilic bacteria such as Thermotoga maritima; thermophilic archaebacteria such as Thermoplasma volcanium; and hyperthermophilic archaeon such as Aeropyrum permix.

WO2000026381A2 discloses a thermostable ligase having 100 fold higher fidelity than T4 ligase and 6 fold higher fidelity than wild-type Thermus thermophilus ligase, when sealing a ligation junction between a pair of oligonucleotide probes hybridized to a target sequence where there is a mismatch with the oligonucleotide probe having its 3′ end abutting the ligation junction at the base immediately adjacent the ligation junction.

WO1994002615A1 describes a thermostable DNA ligase from a hyperthermophilic archeabacterium which catalyzes template-dependent ligation at temperatures of about 30° C. to about 80° C.

US20110053147A1 discloses a modified thermostable DNA ligase having higher DNA binding activity compared to the wild type., which can be obtained by Substituting the negatively charged amino acid(s) present at the N-terminal side of the C-terminal helix moiety of thermostable DNA ligases from thermophilic bacteria, hyperthermophilic bacteria, thermophilic archaea, or hyper thermophilic archaea with non-negatively charged amino acid(s).

WO2004027054A1 describes the characterization of the enzymatic activity of the thermostable TS2126 Rnl1 ligase and its use in RACE protocols.

WO2010094040A1 describes the template-independent intramolecular ligation of linear single-stranded DNA by using a highly adenylated TS2126 Rnl1 ligase with the optional addition of betaine to the ligation reaction mixture.

WO2011123749A1 describes a method for generating adenylated oligonucleotide preparations in an ATP dependent reaction by using a ligase with 90% sequence identity to a ligase obtained from Methanobacterium thermoautotrophicum, Pyrococcus abyssii, phage KVP40, Deinococcus radiodurans, Autographica California, Rhodothermus marinus and phage TS2126.

US20060240451A1 describes methods for ligating linear first-strand cDNA molecules using TS2126 Rnl1 ligase and the amplification of the circular cDNA molecules by rolling circle replication (RCR) or rolling circle transcription (RCT).

U.S. Pat. No. 9,217,167B2 describes methods for the phosphorylation and intramolecular ligation of limited quantities of fragmented chromosomal DNA using TS2126 Rnl1 ligase followed by amplification of the DNA using rolling-circle DNA synthesis. Optimized reaction conditions allow for the multi-step process to function in a single reaction tube without intervening purification steps.

Despite the improvements in template-independent ligation efficiency in the TS2126 Rnl1 ligase, there are some features that are not ideal. These include a template bias since the terminal nucleotides at the end of the single-stranded molecule strongly influences the reaction efficiency, e.g. substrates with 5′-G and 3′-T are circularized most efficiently, while substrates with terminal cytosine bases are very inefficiently ligated (Nunez et al., 2008. Application of Circular Ligase to Provide Template for Rolling Circle Amplification of Low Amounts of Fragmented DNA, The Nineteenth International Symposium on Human Identification, 2008, 7 pages). These also include a slow reaction rate since an efficient ligation requires relatively long incubation times and an excess of enzyme over substrate and that difficult substrates can require very long incubation times and/or the addition of additives such as betaine (Heyer et al., 2015. Nucleic Acids Research, Vol. 43, No. 1, doi: 10.1093/nar/gku1235). Although the highly adenylated form of the ligase exhibits a much higher efficiency of single-stranded DNA circularization than the lowly adenylated form when ATP is omitted from the mixture, efficient reactions require that enzyme be present in a molar concentration greater than the molar concentration of the substrate. In addition, both forms of the ligase display significant differences in intramolecular ligation efficiency using substrates with identical or very similar sizes but with small differences in the sequence composition (WO2010094040A1).

Due to the great importance of ligases and ligation reactions in modern molecular biology and molecular diagnostic methods and assays, there is a great need for improved ligase enzymes to overcome these deficiencies.

### SUMMARY OF THE INVENTION

In order to improve the efficiency and to reduce the template bias in template-independent intramolecular ligation reactions conducted at temperatures high enough to relax single-stranded nucleic acid secondary structures, the inventors have analyzed metagenomic sequencing studies and isolated previously uncharacterized gene products with protein family homology to RNA ligase 1. The identified thermostable ligase candidates showed superior performance compared to the ligase enzymes that are known in the art and that are well established in numerous molecular biological methods and assays such as rolling circle amplification and nucleic acid sequencing library preparation.

The invention relates to an adenylated or unadenylated thermostable ligase consisting of or comprising the amino acid sequence according to SEQ ID NO. 2 or a polypeptide that shares at least 60%, 65%, 70%, 75%, 80%, 85%, 90%, 95% or 98% amino acid sequence identity thereto or a derivative or fragment thereof having a ligase activity.

The invention further relates to a nucleic acid molecule encoding a thermostable ligase consisting of or comprising the nucleic acid sequence according to SEQ ID NO 1 or at least 60%, 65%, 70%, 75%, 80%, 85%, 90%, 95% or 98% nucleic acid sequence identity thereto.

The invention also relates to the use of a thermostable ligase in rolling-circle amplification, rolling-circle transcription, isothermal nucleic acid amplification, amplification of low copy fragmented nucleic acids, sequencing library preparation, attaching RNA and/or DNA adapter sequences to nucleic acid molecules or the like.

Furthermore, the invention relates to a Kit comprising a ligase according to according to SEQ ID NO 2 or a polypeptide that shares at least 60%, 65%, 70%, 75%, 80%, 85%, 90%, 95% or 98% amino acid sequence identity thereto or a derivative or fragment thereof having a ligase activity.

### DETAILED DESCRIPTION OF THE INVENTION

The invention relates to a thermostable DNA and/or RNA ligase enzyme, its amino acid sequence, its nucleic acid sequence and to template DNA and/or RNA ligase proteins encoded by this nucleic acid sequence, as well as nucleic acid or amino acid constructs comprising portions of the nucleic acid or amino acid sequence of the DNA and/or RNA ligase. The thermostable DNA and/or RNA ligase according to this invention allows for an improved and more efficient template-independent intramolecular ligation of single-stranded nucleic acids. The thermostable DNA and/or RNA ligase according to this invention further allows the intermolecular ligation of single-stranded nucleic acids.

Differently from the Rnl1 ligase enzymes known in the art, the ligases according to the present invention were isolated by using a different procedure. Instead of identifying the coding genes based on genetic analysis or DNA sequencing of known organisms, the inventors unexpectedly identified these ligases through very weak homology from environmental metagenomic samples which contain a complex mixture of genes and gene fragments from different organisms and organism types. Because the origin of these genes is unknown and it is unknown whether the genes are expressed or active in vivo, the isolation of a ligase enzyme with the desired activity in vitro was not expected. In fact, the ligases isolated according to the method described herewith showed several advantages over the ligases known in the art, in terms of the lack of template bias and improved reaction kinetics in circularizing single-stranded DNA (see examples 3 and 4). These improved properties are common limitations affecting the efficiency of known RNA ligases in providing complete, efficient single-stranded DNA circular material for various methods of analysis (WO2010094040A1; Nunez et al. 2008. Application of Circular Ligase to Provide Template for Rolling Circle Amplification of Low Amounts of Fragmented DNA). This unique combination of an absence of detectible template bias and fast ligation kinetics on single-stranded DNA with the GBS-3074 ligase allowed the production of much higher yields of circular DNA from small quantities of randomly sheared genomic DNA for rolling-circle amplification and sequencing analysis (see example 5).

Further, the GBS-3074 enzyme has the unique characteristic of being highly active and compatible with a broad range of ATP concentrations in single-stranded circularization reactions (see example 2). Excess ATP is known to compete with adenylated substrate in reactions, thereby causing a “dead end” buildup of intermediate reaction products including adenylated template and adenylated enzyme, which are then incompatible with the DNA end-joining circularization step (Zhelkovsky, A., and McReynolds, L., Nucleic Acids Research, 2011 (39); e117; see WO2010094040A1). To avoid this, the TS2126 enzyme was previously isolated in a highly adenylated form and circularization reactions were conducted in the absence of ATP (see WO2010094040A1). Differently, because the GBS-3074 enzyme is very ATP tolerant, the inventors were able to isolate the enzyme using hydrophobic interaction chromatography in the unadenylated form and then conduct circularization reactions in the presence of high concentrations of ATP. The compatibility with excess ATP and the ability to utilize the unadenylated form of the enzyme is important because it allows for compatibility with carryover ATP from prior enzymatic reactions and for the enzyme to perform multiple rounds of self-adenylation and catalysis in the presence of ATP, which leads to more highly efficient and complete circularization reactions.

It is not at all uncommon especially with virus-derived gene products to show very high levels of sequence divergence between enzymes that are subsequently identified to carry out similar cellular functions. For example, in the case of two previously described thermostable RNA ligase 1 family members derived from the RM378 virus and the TS2126 virus (Blondal et al., 2003. Nucleic Acids Research, Vol 31, No. 24, doi: 10.1093/nar/gkg914; Blondal et al., 2005. Nucleic Acid Research, Vol. 33, No. 1, doi:10.1093/nar/gki149), sequence identity is only 29.3% with each other and show only 24.4% to 29.3% sequence identity with the T4 Rnl1 ligase. In addition, with the previously uncharacterized gene products that the inventors isolated from metagenomic sequences, despite showing well below 60% sequence identity to each other and with RNA ligase 1 family members, these ligases were demonstrated to display single-stranded circularization ligation activity (see Table 3).

The thermostable DNA and/or RNA ligase according to this invention showed several improvements over the DNA and/or RNA ligases that are known in the art and commercially available. Compared to the TS2126 Rnl1 ligase, which is the most improved T4 Rnl1 ligase so far and frequently used in numerous molecular biological and diagnostic applications, it showed a significantly improved reaction rate and thus, it allowed for reduced incubation times and a reduced ligase concentration in the reaction mixture. A reduction of incubation times and thus a reduction of the turn-around-time is one of the key aspects in the development and improvement of molecular diagnostic applications, especially in point-of-care testing, e.g. virus testing. In addition to that, the reduction of reagent concentration and thus a reduction of costs is another key aspect in the development and improvement of modern molecular biological or molecular diagnostic assays.

In contrast to the TS2126 Rnl1 ligase, no template biases, e.g. for substrates having a terminal cytosine base, were observed for the DNA and/or RNA ligase according to this invention. Thus, even for such difficult substrates there was no need for reaction additives such as betaine, bovine serum albumin (BSA), T4 gene 32 protein (gp32) or the like. Such additives often need to be adjusted for any specific assay and sample type and may also have other negative side effects on detection methods, e.g. due to interferences in the fluorescence channels of quantitative PCR cyclers or next generation sequencing apparatuses. Furthermore, these additives are potential sources of inadvertent contamination of molecular detection reagents with residual DNA from expression hosts used for recombinant proteins or from viruses that can be present in materials derived from animal sources such as BSA (Doelger et al., 2020. BioProcess International, Vol. 18, Nr. 4).

Ligases known in the art are very sensitive to adenosine triphosphate (ATP) as shown in WO2010094040A1 for TS2126 Rnl1 ligase. In contrast to that, the unadenylated form of the DNA and/or RNA ligase according to this invention was compatible with a wide range of ATP concentrations and showed nearly complete ligation up to 80 uM ATP. Compatibility with even such high ATP concentrations is an important improvement as it allows for multiple rounds of self-adenylation and catalysis in reaction mixtures containing ATP. In addition, it allowed for compatibility with carryover ATP from prior enzymatic reactions. For example, whereas DNA or RNA molecules that contain a 5′-hydroxyl group are not able to be ligated intermolecularly or intramolecularly, these ends can be phosphorylated by a kinase in reactions that require ATP, converting them to 5′-phosphate ends, which are then able to be ligated. Compatibility with this carryover ATP from the kinase reaction is therefore beneficial because it allows for subsequent ligation without purification of the nucleic acids away from the carryover ATP.

Moreover, the DNA and/or RNA ligase according to this invention showed significantly more efficient ligation of dilute fragmented DNA into amplifiable circular DNA than with TS2126 Rnl1 ligase. This allowed for the more complete, higher quality genetic analysis of samples with low quantities of DNA or fewer starting numbers of cells.

Herein, “ligation” is defined as the joining of two or more nucleic acid fragments, either deoxyribonucleic acid (DNA) molecules and/or ribonucleic acid (RNA) molecules, through the action of an enzyme. Such enzyme may be a ligase enzyme according to this invention.

The term “DNA and/or RNA ligase” means that the ligase enzyme is capable of ligating both single-stranded DNA (ssDNA) fragments and single-stranded RNA (ssRNA) fragments or a combination thereof.

Herein, “thermostable” is defined as a broad range of temperatures in which an enzyme is catalytically active and/or as a high defined unfolding or transition temperature or melting temperature or if a long half-life at a selected broad range of temperatures is observed.

Herein, the term “template-independent ligation” is defined as an intermolecular and/or intramolecular ligation of linear ssDNA and/or ssRNA in the absence of a ligation template, such as a target nucleic acid, bridging or a splint nucleic acid molecule to which the ends of the linear ssDNA and/or ssRNA that one desires to ligate can anneal so that its ends are adjacent.

Herein the term “bridging or splint nucleic acid molecule” is defined as a nucleic acid molecule, in particular a DNA and/or RNA oligonucleotide, that is hybridized to the ssDNA and/or ssRNA molecules, which shall be ligated, prior to ligation; e.g. in order to tether them in the correct orientation.

Herein the term “intramolecular ligation” means the joining of both ends of a ssDNA and/or a ssRNA molecule that results in the circularization of such molecule, whereas the term “intermolecular ligation” means the joining of two or more ssDNA and/ssRNA molecules. A ssDNA and/or ssRNA molecule that has been generated by joining two or more ssDNA and/or ssRNA molcules by intermolecular ligation, may be circularized by intramolecular ligation.

Herein the terms “circularized ssDNA and/or ssRNA” or “circularization of a ssDNA and/or ssRNA molecule” mean that such molecule has formed a covalently closed loop structure. Circularized ssDNA and/or ssRNA molecules inter alia show a higher resistance to exonuclease degradation, better thermodynamic stability and the capability of being replicated in a rolling circle manner by DNA polymerases.

Herein the term “reaction rate” means the speed at which the ligase enzyme converts ssDNA and/or ssRNA substrates into intramolecular and/or intermolecular ligated products. Usually, the reaction rate is highly dependent upon ligase enzyme concentration and incubation time.

The present invention relates to a novel thermostable ligase enzyme consisting of or comprising an amino acid sequence according to SEQ ID NO 2, referred to as GBS-3074 ligase, that was found to catalyze the template-independent intramolecular and/or intermolecular ligation of either ssDNA and/or ssRNA.

Unexpectedly, the inventors saw in experiments that the GBS-3074 ligase also showed intermolecular ligation activity. Although the intramolecular ligation activity (circularization) was the main focus of the inventors, the capability of intermolecular ligation under the appropriate reaction conditions is another important characteristic of the GBS-3074 ligase.

The GBS-3074 ligase was thermostable up to 75° C. and showed ligation activity up to this temperature. This broad range of thermostability is useful in various nucleic acid techniques known to those skilled in the art and as set forth herein.

The thermostable single-stranded DNA and/or RNA ligase according to the invention, referred to as GBS-3074 ligase, can be used at a temperature in the range of 45° C. to 75° C., preferably in the range of 55° C. to 70° C., more preferably at 60 to 65° C.

The thermostable single-stranded DNA and/or RNA ligase according to the invention, referred to as GBS-3074 ligase, can be used at a pH in the range of pH 6.5 to pH 8.0, preferably in the range of pH 7.0 to pH 8.0, more preferably at pH 7.5.

The invention relates to a thermostable DNA and/or RNA ligase consisting of or comprising an amino acid sequence according to SEQ ID NO 2, SEQ ID NO 4 or SEQ ID NO 6 or a polypeptide that shares at least 60%, 65%, 70%, 75%, 80%, 85%, 90%, 95% or 98% amino acid sequence identity thereto or a derivative or fragment thereof having ligase activity.

The invention further relates to thermostable DNA and/or RNA ligase consisting of or comprising an amino acid sequence according to SEQ ID NO 2, SEQ ID NO 4 or SEQ ID NO 6 or a polypeptide that shares at least 60%, 65%, 70%, 75%, 80%, 85%, 90%, 95% or 98% amino acid sequence identity thereto or a derivative or fragment thereof having a ligase activity, wherein the ligase is capable of intermolecular ligation of two or more RNA and/or DNA molecules or intramolecular ligation of an RNA or DNA molecule, wherein the RNA or DNA molecule may be single-stranded.

The ligases disclosed in US20040259123A1, US20090061481A1, WO02000026381A2, WO1994002615A1 and US20110053147A1 originates from archaeal species (in particular, the Crenarchaea A. pernix for US20040259123A1 and Pyrococcus furiosus for US20090061481A1, WO1994002615A1 and US20110053147A1) and are identified as ATP-dependent DNA ligases. The ligase described in WO2000026381A2 is from Thermus sp. AK16D and is very similar to Taq ligase, a thermostable NAD-dependent DNA ligase. The activities described for these ligases are also consistent with other DNA ligases: they are catalyzing cohesive end joining of double-stranded DNA molecules and nick sealing on double-stranded DNA. These ligases operate in a template-dependent manner using a bridging or splint DNA molecule. Thus, these ligases may only be capable of intramolecular ligation of double-stranded DNA in a template-dependent manner using a bridging or splint DNA molecule.

Differently, the ligases according to the present invention allow the circularization of single-stranded DNA or RNA molecules in a template-independent procedure.

The invention further relates to thermostable DNA and/or RNA ligase, wherein the ligase does not require a bridging or splint nucleic acid molecule for ligation.

The invention also relates to a nucleic acid molecule encoding a thermostable DNA and/or RNA ligase as described herein consisting of or comprising a nucleic acid sequence according to SEQ ID NO 1, SEQ ID NO 3 or SEQ ID NO 5 or at least 60%, 65%, 70%, 75%, 80%, 85%, 90%, 95% or 98% nucleic acid sequence identity thereto.

The invention also pertains to an expression vector containing a nucleic acid sequence as described above encoding a thermostable DNA and/or RNA ligase enzyme as described herein or an active derivative or fragment thereof, operably linked to at least one regulatory sequence. Many expression vectors are commercially available, and other suitable vectors can be readily prepared by the skilled artisan. Regulatory sequences are known in the art and are selected to produce the polypeptide or active derivative or fragment thereof.

Herein, the term “operably linked” is defined as that the nucleotide sequence is linked to a regulatory sequence in a manner which allows expression of the nucleic acid sequence.

Herein, the term “regulatory sequence” means promoters, enhancers, and other expression control elements as described in the literature (e.g. Goeddel (1990), Gene Expression Technology: Methods in Enzymology, Vol. 185, Academic Press, San Diego, CA).

The GBS-3074 ligase has a sequence identity of approximately 30% compared to the TS2126 Rnl1 ligase.

Surprisingly, the inventors found that the GBS-3074 ligase shows no template bias (see Example 2) and a highly improved ligation efficiency over the TS2126 Rnl1 ligase (see Examples 3 and 4).

Herein, the term “template bias” means that the circularization efficiency differs when substrates with different terminal nucleotides are circularized, e.g. when the ligase displays preferences for certain substrates such as those that contain 5′-G and 3′-T, while certain substrates, such as those containing terminal cytosine bases, are ligated inefficiently.

Herein the term “ligation efficiency” is defined as percentage of ligation products relative to the amount of initial ligation substrates over time, e.g. the ligation efficiency is higher in case if 80% of the substrates are circularized by intramolecular ligation after 30 minutes reaction time than if only 25% of the substrates are circularized in the same time or if it takes for example 75 minutes reaction time to reach the 80% of circular products relative to the amount of initial ligation substrates.

The inventors found that two forms of the GBS-3074 ligase could be separated using hydrophobic interaction chromatography, which were identified as a primarily self-adenylated form and an unadenylated form. In the first step of the well-known three-step mechanism of ligase catalysis (Pascal, 2008. Current Opinion in Structural Biology, Vol. 18, Nr. 1, doi: 10.1016/j.sbi.2007.12.008), an enzyme-adenylate intermediate is formed after reaction of the ligase with adenosine triphosphate (ATP). Activity tests of the two forms of GBS-3074 ligase showed that while the adenylated form did not require ATP in the reaction buffer and was inhibited by ATP, the unadenylated form absolutely required ATP for activity (FIG. 1). The unadenylated form of the enzyme was chosen as the preferred form because the presence of ATP in the reaction mix allows for multiple rounds of self-adenylation and catalysis.

The invention relates to a thermostable DNA and/or RNA ligase that is predominantly unadenylated.

The invention further relates to a thermostable DNA and/or RNA ligase that is predominantly unadenylated and requires ATP for activity.

The invention also relates to a thermostable DNA and/or RNA ligase that is predominantly adenylated.

The invention further relates to a thermostable DNA and/or RNA ligase that is predominantly adenylated and inhibited in the presence of ATP.

Most of the commercially available ligases show a template bias which can be a severe issue in molecular biological and molecular diagnostic assays. To determine whether the GBS-3074 ligase displays template bias, a preference for particular terminal nucleotides at the ends of the single-stranded molecule, 10 different substrates were tested for ligation efficiency (FIG. 2). Unexpectedly, it was found that all substrates were circularized nearly to completion in 60 minutes or less. In contrast, in agreement with previous reports (Nunez et al., 2008. Application of Circular Ligase to Provide Template for Rolling Circle Amplification of Low Amounts of Fragmented DNA, The Nineteenth International Symposium on Human Identification, 2008, 7 pages) it was found that the TS2126 Rnl1 ligase displayed marked preferences for certain substrates such as those that contain 5′-G and 3′-T, while certain substrates were ligated inefficiently, such as those containing terminal cytosine bases (FIG. 3).

Thus, the invention also relates to a thermostable DNA and/or RNA ligase that is capable of catalyzing ligation reactions that are predominantly free of any template bias.

Herein, “predominantly free of any template bias”, means that substrates with specific terminal nucleotides are not preferentially ligated over others.

Decreased concentration of ssDNA and/or ssRNA substrate and increased ssDNA and/or ssRNA fragment lengths can both have negative impact on the rate of intramolecular circularization because of decreased effective concentration of ssDNA and/or ssRNA ends available for catalysis by the ligase enzyme (cf. Shore et al., 1981. PNAS, Vol. 78, Nr. 8, doi: doi.org/10.1073/pnas.78.8.4833). The inventors found that the GBS-3074 ligase showed dramatic improvements over existing commercially available ligases regarding the intramolecular ligation activity with both increased fragment lengths of about 200 bp and decreased ligation substrate concentrations.

Therefore, the invention relates to a thermostable single-stranded DNA and/or RNA ligase that is capable of intramolecular ligation of random substrates of lengths of about 200 bp that are present in quantities of 1 ng or more to less than 100 fg (see Example 5).

The invention further relates to a thermostable single-stranded DNA and/or RNA ligase that is capable of intramolecular ligation of substrates with a length of about 50 nucleotides or less up to substrates with a length of 200 nucleotides or more in a template-independent manner.

Moreover, the inventors unexpectedly found that the ligation kinetics on such substrates are much faster with GBS-3074 ligase compared to the TS2126 Rnl1 ligase (FIG. 4 and FIG. 5a), indicating that shorter reaction times can be used to achieve efficient ligation of all substrate types.

The thermostable DNA and/or RNA ligase enzyme according to this invention can be utilized in methods such as, but not limited to: rolling-circle amplification, digital nucleic acid analysis (e.g. digital PCR or digital droplet PCR), rolling-circle transcription, isothermal nucleic acid amplification methods, amplification of low copy fragmented DNA for forensic applications, sequencing and next generation sequencing library preparation workflows, whole genome sequencing, whole-genome bisulfite sequencing, amplifying cDNA ends for random amplification of cDNA ends (RACE), 3′ end labeling of RNA, oligonucleotide synthesis, cDNA adapter ligation, rapid amplification of cDNA ends (RLM-RACE), ligation of single-stranded primer products for PCR and many more that are known to those skilled in the art.

Additionally, the invention relates to a kit containing a thermostable template-independent DNA and/or RNA ligase as described herein or to a kit comprising a thermostable template-independent DNA and/or RNA ligase as described herein, and optionally, a buffer and/or oligonucleotides.

The references cited herein are incorporated by reference in their entirety. The invention has been shown and described with references to preferred embodiments thereof. The invention will be understood by those skilled in the art that various changes in form and details may be made therein without departing from the scope of the invention as defined by the claims.

### EXAMPLES

### Example 1: Identification of a Highly Active Thermostable Single-Stranded Ligase

In an effort to identify highly active and thermostable ligases capable of template-independent DNA and RNA circularization, searches were conducted for previously uncharacterized gene products with protein family homology to T4 Rnl1 in a database containing sequences from metagenomic sampling studies, the Joint Genome Institute Integrated Microbial Genomes and Microbiomes system (https://img.jgi.doe.gov/; Chen et al., 2019. Nucleic Acid Research, Vol. 47, Nr. D1, doi: 10.1093/nar/gky901). After limiting the results to those studies in which sampling was conducted at a geographic location in which thermophilic organisms would be expected to grow, a list of 13 viral gene products was generated (Table 1). Each of these DNA sequences was codon optimized for expression in E. coli, and the corresponding synthetic gene fragments were constructed and assembled into an expression vector. After sequence verification, ligases were overexpressed in BL21 cells. Of the original 14 candidates, 8 showed detectable protein expression and 6 of these produced soluble protein that was then purified by iterative rounds of affinity and ion exchange chromatography. To measure for high temperature template-independent ligation activity, a single-stranded 64 nucleotide 5′-phosphorylated oligonucleotide substrate according to SEQ ID NO 7 (5′-/5phos/gtctggttggtcagccgttgtgggatgttagccgtagcagcactggtaatctggttgaatggtt) was reacted with each of the ligases and the extent of conversion of the linear form to the circular form was determined using denaturing polyacrylamide gel electrophoresis. Reactions (20 μl ) containing 33 mM HEPES-KOH, pH 7.5, 66 mM KOAc, 0.5 mM DTT, 2.5 mM MnCl₂, 50 μM ATP, 0.5 μM oligonucleotide substrate, and 2 μM enzyme were incubated at 55° C. for 1 hour. Linear and circular DNA products were fractionated by electrophoresis using 15% polyacrylamide Tris-borate-EDTA gels containing 7 M urea (TBE-urea), then gels were stained with 2X SYBR Gold (Invitrogen) and band intensities were quantified. It was found that 3 of the candidates showed detectable activity and one of these, referred to as GBS-3074 ligase (locus tag Ga0072500_1423074, SEQ ID NO 1 and SEQ ID NO 2) showed high levels of activity, converting nearly all of the substrate to the circular form. Unlike better characterized DNA ligases, because the GBS-3074 ligase gene was sequenced as part of a large metagenomic study containing a complex mixture of genes and gene fragments from many organisms and organism types in an environment, it is unknown whether the ligase gene is expressed in vivo, from which virus this gene originates, and what species or type of cell the originating virus infects.

Table 1 below depicts the putative thermophilic Rnl1 enzymes from metagenomic Rnl1 genes that were synthesized, expressed, and screened for template-independent intramolecular DNA ligation. The percent identity relative to the TS2126 Rnl1 ligase is shown. Percent coverage indicates the portion of the candidate protein used in the BLAST alignment to measure identity and similarity. The candidate in bold type (locus tag Ga0072500_1423074; SEQ ID NO. 1 and SEQ ID NO 2) corresponds to the most active ligase, referred to as GBS-3074 ligase. The candidates in italics (locus tag Ga0209741_10051251, SEQ ID NO. 5, SEQ ID NO.6; locus tag Ga0105160_10035846; SEQ ID NO. 3 and SEQ ID NO. 4) did show some single-stranded circularization activity as well.

TABLE 1
List of thermophilic Rnl1 enzymes obtained from metagenomic Rnl1 genes

| | | Size | | |
|---|---|---|---|---|
| | | (amino | Percent | Coverage |
| Locus Tag | Geographic Location | acids) | Identity | (%) |
| GBSCSSed85CDRAFT_0027843 | Great Boiling Spring, Nevada | 372 | 32 | 49 |
| Ga0105154_10074901 | USA: Nevada, Gerlach, | 374 | 32 | 49 |
| | Sandy's Spring West | | | |
| Ga0209394_100018137 | Baoshan, Yunnan, China | 387 | 34 | 29 |
| Ga0072500_1423074 | Great Boiling Spring, Nevada | 385 | 31 | 46 |
| Ga0072500_1437516 | Great Boiling Spring, Nevada | 374 | 32 | 49 |
| JzSedJan11_100158159 | Baoshan, Yunnan, China | 417 | 34 | 29 |
| Ga0065719_1120151 | Great Boiling Spring, Nevada | 313 | 32 | 49 |
| Ga0105154_10074901 | USA: Nevada, Gerlach, | 374 | 32 | 49 |
| | Sandy's Spring West | | | |
| Ga0072500_{—}1423074 | Great Boiling Spring, Nevada | 384 | 31 | 46 |
| Ga0072500_1437516 | Great Boiling Spring, Nevada | 374 | 32 | 49 |
| JzSedJan11_100158159 | Baoshan, Yunnan, China | 417 | 34 | 29 |
| Ga0209741_{—}10051251 | Octopus Spring, Yellowstone | 382 | 31 | 38 |
| Ga0105160_{—}10035846 | China: Gongxiaoshe hot spring | 425 | 29 | 90 |

### Example 2: Separation and Characterization of Adenylated and Unadenylated Forms of GBS-3074 Ligase.

During purification of the GBS-3074 ligase from E. coli lysate, it was noted that two forms of the protein could be separated by phenyl sepharose hydrophobic interaction chromatography using HiTrap Phenyl HP columns (Cytiva Life Sciences), which were subsequently identified as a primarily self-adenylated form and an unadenylated form. In the first step of the well-known three-step mechanism of ligase catalysis, an enzyme-adenylate intermediate is formed after reaction of the ligase with ATP. Activity tests of the two forms of GBS-3074 ligase showed that whereas the adenylated form did not require ATP in the reaction buffer and was inhibited by ATP (FIG. 1a), the unadenylated form absolutely required ATP for activity (FIG. 1b). The GBS-3074 ligase showed high circularization activity in the presence of a wide range of ATP concentration from 0.63 μM to 80 μM, whereas TS2126 Rnl1 ligase circularization activity was inhibited in the presence of 12.5 μM ATP and showed significant inhibition in the presence of 50 μM ATP. These reactions (20 μl ) contained 33 mM HEPES-KOH, pH 7.5, 0.5 mM DTT, 2.5 mM MnCl₂, 0.5 μM single-stranded substrate, 0.5 μM ligase, the indicated quantity of ATP, and were incubated at 55° C. for 1 hour. The oligonucleotide substrate was a 5′-phosphorylated 64 nt oligonucleotide according to SEQ ID NO 8 with both a 5′ and 3′ terminal adenosine base (5′-/5phos/atctggttggtcag ccgttgtgggatgttagccgtagcagcactggtaatctggttgaatggta). Linear and circular DNA products were fractionated by electrophoresis using 15% polyacrylamide TBE-urea gels, then stained with 2X SYBR Gold (Invitrogen). The unadenylated form of the enzyme was chosen as the preferred form for further characterization because of its ability to catalyze more complete substrate circularization, the wider range in tolerance to higher ATP concentrations, and the potential for multiple rounds of self-adenylation and catalysis using the unadenylated ligase in reaction mixtures containing ATP. In addition, tolerance to ATP allows compatibility with carryover ATP from prior enzymatic reactions.

For example, ends of a DNA or RNA molecule can be phosphorylated by a kinase in reactions that require ATP, converting them to 5′-phosphate ends, which are then able to be ligated. Compatibility with this carryover ATP from the kinase reaction would therefore be beneficial because it would allow subsequent ligation without purification of the nucleic acids away from the carryover ATP.

### Example 3: Circularization Efficiency Using Substrates with Different Terminal Nucleotides

To determine whether the GBS-3074 ligase displays template bias, a preference for particular terminal nucleotides at the ends of the single-stranded molecule, 10 different substrates were tested for ligation efficiency (FIG. 2). These substrates were all 5′-phosphorylated oligonucleotides according to SEQ ID NO 9 (5′-/5phos/ntctggttggtcagccgttgtgggatgttagccgtagcagca ctggtaatctggttgaatggtn), where n=a, g, c or t. Reactions (20 μl ) containing 33 mM HEPES-KOH, pH 7.5, 0.5 mM DTT, 2.5 mM MnCl₂, 50 μM ATP, 0.5 μM oligonucleotide substrate, and 1 μM unadenylated GBS-3074 ligase were incubated at 55° C. Reactions containing CircLigase II (Lucigen) were reacted with the same substrates at 55° C. using the manufacturer recommended conditions. Linear and circular DNA products were fractionated by electrophoresis using 15% polyacrylamide TBE-urea gels, then stained with 2XSYBR Gold (Invitrogen) and the band intensities were quantified. It was found that all substrates were circularized by the unadenylated GBS-3074 ligase nearly to completion (FIG. 2). In contrast, it was found that the TS2126 Rnl1 ligase displayed marked preferences for certain substrates such as those that contain 5′-G and 3′-T, while certain substrates were ligated inefficiently, such as those containing terminal cytosine bases (FIG. 3).

### Example 4: Measurement of Single-Stranded DNA Circularization Reaction Kinetics

To determine the relative speed at which the GBS-3074 ligase catalyzed circularization of single-stranded DNA substrates with different terminal nucleotides, a time-course reaction was performed with a fluorescently labeled substrate oligonucleotide and products were analyzed by denaturing capillary electrophoresis (FIG. 4). GBS-3074 ligase reactions (15 μl ) contained 33 mM HEPES-KOH, pH 7.5, 0.5 mM DTT, 2.5 mM MnCl₂, 25 μM ATP, 0.5 μM oligonucleotide substrate, and μM unadenylated ligase. CircLigase II reactions (15 μl ) contained 1 μM ligase, 0.5 μM oligonucleotide substrate, 2.5 mM MnCl₂ and the manufacturer recommended buffer. Oligonucleotide substrates had an internal fluorescein-labeled thymine base on position 28 and had the sequence according to SEQ ID NO 10 (5′-/5phos/gtctggttggtcagccgtt gtgggatgntagccgtagcagcactggtaatctggttgaatggtg) (FIG. 4a) or SEQ ID NO 11 (5′-/5phos/ctctg gttggtcagccgttgtgggatgntagccgtagcagcactggtaatctggttgaatggtc) (FIG. 4b), where n=internal fluorescein-labeled thymine at position 28. Reactions were incubated at 55° C. for the indicated time, then stopped by adding 1 μl of a solution of 200 mM EDTA and 3.2 mg/ml proteinase K, followed by an additional incubation at 37° C. for 30 minutes. Products were analyzed by capillary electrophoresis in formamide on a 3730xl DNA Analyzer (Applied Biosystems). Peaks corresponding to linear oligonucleotide (substrate) and circularized single-stranded DNA (ligation product) were quantified and the percentage circular product was plotted. Virtually no concatemerized end-to-end ligation products were detected. It was found that the circularization ligation kinetics and extent of ligation were similar between the two ligases using the substrate with the 5′-G and 3′-G nucleotides (FIG. 4a), except for a slightly longer lag phase at early time points with CircLigase II. However, using the substrate with 5′-C and 3′-C nucleotides, the GBS-3074 ligase showed much faster ligation kinetics than CircLigase II (FIG. 4b), with a ligation rate and extent of ligation similar to the reactions with the 5′-G/3′-G substrate. In contrast, the reactions catalyzed by CircLigase II only reached as much as 24% completion after 60 minutes on the 5′-C/3′-C substrate. The low template bias with GBS-3074 ligase indicates that shorter reaction times can be used to achieve efficient ligation of many substrate types and improve the reaction uniformity.

### Example 5: Single-Stranded Circularization Activity Using Larger, Randomly Fragmented Genomic DNA Fragments

Decreased concentration of DNA substrate and increased DNA fragment lengths can both have negative impacts on the rate of intramolecular circularization because of decreased effective concentration of DNA ends available for catalysis by the DNA ligase. In order to demonstrate the capability of the GBS-3074 ligase for circularizing very diluted DNA fragments with a range of lengths, a substrate was prepared by randomly shearing E. coli genomic DNA to an average size of 200 bp using focused ultrasonication (Covaris). These fragments were composed of a random mixture of sequences with all possible combinations of terminal nucleotides. For GBS-3074 ligase, ligation reactions (10 μl ) contained 33 mM HEPES-KOH, pH 7.5, 0.5 mM DTT, 2.5 mM MnCl₂, 25 μM ATP, sheared E. coli DNA, and 1 μM unadenylated ligase. CircLigase II reactions (10 μl ) contained 1 μM ligase, sheared E. coli DNA, 2.5 mM MnCl₂ and the manufacturer recommended buffer. Reactions were assembled without ligase, heated at 95° C. for 3 minutes to separate the fragmented E. coli genomic DNA into single-strands, then rapidly transferred to an ice block. Reactions were initiated by adding ligase and were incubated for 1 hour at 60° C. for both GBS-3074 ligase reactions and CircLigase II reactions. Circularized DNA products were amplified by Phi29-mediated rolling circle amplification by adding 2.3 μl to reactions (15 μl ) containing 50 mM HEPES, pH 8.0, 20 mM MgCl₂, 0.01% Tween-20, 2 mM DTT, 20 mM KCl, 40 μM phosphorothioated random hexamer, 0.5X SYBR Green I (Invitrogen), 0.4 mM dNTPs, and 20 μg/ml Phi29 polymerase. Incubation was at 30° C. for 4 hours in a StepOnePlus system (Applied Biosystems), with fluorescence readings taken every minute. For each reaction, a threshold time was determined by measuring the time at which the fluorescence reading reached 60,000 relative fluorescence units and results were plotted against DNA input quantity on a semi-log scale. Whereas reactions without ligase showed very long threshold times of generally 120 minutes or more because of inefficient multiple displacement amplification, reactions treated with a single-stranded ligase showed faster threshold times, indicating a conversion to a rolling-circle mode of amplification (FIG. 5a). At 1 ng of template DNA input the threshold time of the GBS-3074 ligase ligation reaction was only a small amount shorter than that of the CircLigase™ II ligation reaction, but at input quantities smaller than this, GBS-3074 ligase reactions showed a substantially shorter threshold time, indicating significantly more efficient ligation of dilute fragmented DNA into amplifiable circular DNA.

To analyze the sequence content of the rolling circle-amplified DNA, reactions products were purified and processed into Illumina sequencing libraries. After heat inactivation of the Phi29 polymerase at 65° C. for 10 minutes, DNA was ethanol precipitated, washed, and resuspended in 10 mM Tris, pH 8.0. DNA yields were generally in the range of 4-6 μg. To generate the sequencing libraries, 500 ng of amplified DNA was fragmented, end polished, and ligated to adapters using the sparQ DNA Frag & Library Prep Kit (Quantabio) without additional PCR amplification. Libraries were then pooled and sequenced using the MiSeq 2X150 paired end protocol (Illumina), and then the resulting read quantity was normalized by random sampling of 1.75 million reads for each sample. Mapping to the E. coli reference genome showed that GBS-3074 ligase ligation reactions were sufficiently efficient to allow recovery of greater than 95% of the genomic DNA sequences even with an input quantity of 10 pg (FIG. 5b). In contrast, CircLigase II reactions showed only 51% genome coverage from ligation reactions using 100 pg fragmented DNA input and only 13% genome coverage from 10 pg ligation input reaction. In addition, the median genome coverage levels of the E. coli reference DNA sequence were significantly higher from those ligation reactions using GBS-3074 ligase for circularization of the fragmented input DNA (FIG. 5c).

### Example 6: Circularization of Single Stranded DNA and RNA Substrates

To determine the substrate compatibility of the GBS-3074 ligase with single-stranded RNA nucleic acid templates, circularization reactions were performed with both a 64 nt DNA oligonucleotide and a 56 nt RNA oligonucleotide with the same terminal base composition (FIG. 6). These reactions (20 μl ) contained 33 mM HEPES-KOH, pH 7.5, 0.5 mM DTT, 2.5 mM MnCl₂, 0.5 μM single-stranded substrate, 1 μM ligase, 1.25 μM ATP, and were incubated at 55° C. for 1 hour. The DNA oligonucleotide substrate was a 5′-phosphorylated oligonucleotide according to SEQ ID NO 12 (5′-/5phos/ttctggttggtcagccgttgtgggatgttagccgtagcagcactggtaatctggttgaatggtc) and the RNA substrate was a 5′-phosphorylated RNA oligonucleotide according to SEQ ID NO 13 (5′-/5phos/uagg cgucggugacaaacggccagcguuguugucucucuguucuagcuuaucgguc). Linear and circular DNA or RNA products were fractionated by electrophoresis using 15% polyacrylamide TBE-urea gels, then stained with 2X SYBR Gold (Invitrogen). Both substrate types were circularized nearly to completion indicating that GBS-3074 ligase is compatible with both DNA and RNA.

### Example 7: Characterization of GBS-3074 Ligase Optimal Reaction Temperature and pH

To determine the thermal compatibility of the GBS-3074 ligase and optimal reaction temperature, circularization reactions were performed with a 5′-phosphorylated 64 nt DNA oligonucleotide substrate according to SEQ ID NO 14 (5′-/5phos/ctctggttggtcagccgttgtgggatgttag ccgtagcagcactggtaatctggttgaatggtc) and reactions were incubated at temperatures ranging from 45° C. to 75° C. as indicated (FIG. 7a). These reactions (15 μl ) contained 33 mM HEPES-KOH, pH 7.5, 0.5 mM DTT, 2.5 mM MnCl₂, 0.5 μM single-stranded substrate, 0.5 μM ligase, 25 μM ATP, and were incubated for 15 minutes. Linear and circular DNA products were fractionated by electrophoresis using 15% polyacrylamide TBE-urea gels, stained with 2X SYBR Gold (Invitrogen), then band intensities were quantified and the percentage of circularized substrate was calculated and plotted. While significant activity was observed throughout the entire range of temperatures from 45° C. to 75° C., the optimal circularization reaction temperature for GBS-3074 ligase was observed to be 60° C. to 65° C.

The optimal reaction pH for the GBS-3074 ligase was determined by performing DNA circularization reactions in which HEPES-KOH buffer pH was varied from 7.0-8.0 (FIG. 7b). Reactions (20 μl ) contained 33 mM HEPES-KOH, 0.5 mM DTT, 2.5 mM MnCl₂, 0.5 μM single-stranded substrate (SEQ ID NO 13), 1 μM ligase, 1.25 μM ATP, and were incubated for either 30 or 60 minutes. At both time points the largest quantity of circularized DNA product was observed at pH 7.5, indicating the fastest reaction rate at this pH value.

TABLE 2
Amino acid and nucleic acid sequences

| | |
|---|---|
| SEQ ID NO 1 | 5′atgacgttatatgagttacgtaagggcttagaagcggtgaaacattacatccttaacaacgacgca |
| (DNA sequence of a | tcagcgtcggggtataacgacgacttactggagcgtatggtatgggtgaatcacgaatatgatctggt |
| thermostable ligase | cctgttaaactacaaagatgccactgctatgatcctgcataacgaagggttacagtggacacctttttt |
| enzyme referred to | gcgtgtttgtcgtggggttgtatttacgccttcaggcgaactggtctcgttgcccttacacaaattcttca |
| as GBS-3074 ligase. | atgtaaaggagaacgaagagacctcgttggctaatatcgctaattggcctctgcgtagtgctaccgag |
| Locus tag | aaggtagacggggtcatgattcaggtgttcttccacccactgcgcaaggaaattacctatgccagtcg |
| Ga0072500_1423074) | ctggcgcatttggtccgacgcagccatcactgcgtttaaattagcgaactcagcgctgactaacgcgg |
| | taatcccaaagctgaatgcctctttcggtgaaggaaagtggacgttaatttgtgaattaatccatccag |
| | agcatcgtcagcccggaatggttagctatggggacttacaagcgttggtgcttctgtacgtccgcaaat |
| | tagacgatcttgagttgattccagccgtagaattgtttaaggataacgaattgccgcccccacttatgc |
| | tgccacagcaatatttaattgtgtccgcactggaagcccttgagaaagtcaagcaggccaagcacgc |
| | gaattgggagggtattgttgttcagggggcaatggagggtgggaatcgtctggtgaagatgaaaaac |
| | cctctgtacttagagggagtaaacgccgtgaaaaacttgaatcgcattttaaagatctatgaagcaca |
| | ggggcgcgaaggcgtggaaaacctgttcctgctgtacgcatcctacttggacgatgtcccgcacatcg |
| | tgggtcttcgcgatttgttgtacaagaccgaggacgagattaacaactacgctaagcagttgcgcgaa |
| | tcgacacaggacgtgactaccttgcctcgtgaatggcgttgggtcaaatcttatgacgtcggcaatga |
| | caaatggcagcgctgcgtccgccgtatggtactgcaaaaagtgaacgcaggcggtcgtaagtaa 3′ |
| SEQ ID NO 2 | MTLYELRKGLEAVKHYILNNDASASGYNDDLLERMVWVNHEYDLVLLNYKDAT |
| (Protein sequence of | AMILHNEGLQWTPFLRVCRGVVFTPSGELVSLPLHKFFNVKENEETSLANIANW |
| a thermostable | PLRSATEKVDGVMIQVFFHPLRKEITYASRWRIWSDAAITAFKLANSALTNAVIPK |
| ligase enzyme | LNASFGEGKWTLICELIHPEHRQPGMVSYGDLQALVLLYVRKLDDLELIPAVELFK |
| referred to as GBS- | DNELPPPLMLPQQYLIVSALEALEKVKQAKHANWEGIVVQGAMEGGNRLVKM |
| 3074 ligase. Locus tag | KNPLYLEGVNAVKNLNRILKIYEAQGREGVENLFLLYASYLDDVPHIVGLRDLLYKT |
| Ga0072500_1423074) | EDEINNYAKQLRESTQDVTTLPREWRWVKSYDVGNDKWQRCVRRMVLQKVNAGGRK |
| SEQ ID NO 3 | 5′atggaagagcgggtccgtgtttatcaagctataccatccctggaacgcgcgtttgacatagctaaa |
| (DNA sequence of a | gacgctaaggccatagcgtttcgtacctcggaagaaggacttgtattatttaactatttgttttctgacc |
| thermostable ligase | aacagctgtggacacaggtacccgagtcgcgtaacttgagaggtattgtctatgagcaaacgtctgg |
| enzyme. Locus tag | acgggtggtctctctgcccttccataagttttttaacccgggggagccagcttctccggacgtttcaaaa |
| Ga0105160_10035846) | tacgattttggaaagtcacttgtctccaaaaagcacgatggatacttgctgcagacgtttgtgtaccgt |
| | gggaaagtctacactatctccagacactcttttaaggctccactggttcaaacagtcttacagggctta |
| | tgggacaagcgccacgaacgttttgtattacaggtctctgaggagtatccgcagggaattacactgtt |
| | gtgggaagttatacatcccctttatccagtcctggaacttccagaaaagccttcactggtcttattagct |
| | gctcgcatgaccgacacaggggattacttattccccgtaatagagggagaatctgacccaccgtttga |
| | ggttaaaacgctgtcagtacctagtagtttcttatcagatggaatcacggaggtagcccgttggcttcc |
| | cgtgtctagtcttttcgaaaattactcgtcctggaacgacataaaacagcaggtcaagtcaattcaccg |
| | ctctgaaggatatgttatagcgttgtttacacaagaggggtccgaattcgtttttgacgatttcgtaaaa |
| | gctaagactccttgggcgttcaaagcgtccttgttattcgctaaccctggggatacgcttgtgcgttcag |
| | tggtcgaggataaggtggatgatcttgtgtacgaggtgttaaaagatgatccgcgtctgcaagcattt |
| | agtaaggcccacagaacgttgttaaaccacatctatcttgcctacgatttcggtcttggtctgagtcaa |
| | aagcaggtcgaagcgaaagatgcttaccaggccgccgtgtcatggtcacagccctacggtaaatacc |
| | acccagagttgccgagcgtatttaccccgttgataatgaaggcctaccgcggtgcgtcctttgaagaa |
| | gtttgggagaatttcaaaaaattaatggagaacaaaaaaaagttggttgcagtttctagctggattga |
| | acttacccatcaacttcactacgtggagccagatgga 3′ |
| SEQ ID NO 4 | MEERVRVYQAIPSLERAFDIAKDAKAIAFRTSEEGLVLFNYLFSDQQLWTQVPES |
| (Protein sequence of | RNLRGIVYEQTSGRVVSLPFHKFFNPGEPASPDVSKYDFGKSLVSKKHDGYLLQTF |
| a thermostable | VYRGKVYTISRHSFKAPLVQTVLQGLWDKRHERFVLQVSEEYPQGITLLWEVIHP |
| ligase enzyme. Locus | LYPVLELPEKPSLVLLAARMTDTGDYLFPVIEGESDPPFEVKTLSVPSSFLSDGITEV |
| tag | ARWLPVSSLFENYSSWNDIKQQVKSIHRSEGYVIALFTQEGSEFVFDDFVKAKTP |
| Ga0105160_10035846) | WAFKASLLFANPGDTLVRSVVEDKVDDLVYEVLKDDPRLQAFSKAHRTLLNHIYL |
| | AYDFGLGLSQKQVEAKDAYQAAVSWSQPYGKYHPELPSVFTPLIMKAYRGASFE |
| | EVWENFKKLMENKKKLVAVSSWIELTHQLHYVEPDG |
| SEQ ID NO 5 | 5′atgactattcagcaactgcgcgatggattagcccaagtaatggagttcgtgcgcaagaaccagtac |
| (DNA sequence of a | ccttcggaaatcggtcgttattttatccgccgtcgctgggaaaatttagtcctgttaaactacgctgact |
| thermostable ligase | cagccgtttacaaattctcggcagacgagtggacgccgccgatgcgtgtttgtcgcggagtgatcgta |
| enzyme. Locus tag | acggatgacggatcacaagtggtcagctttcctttccataaattttttaatgttggggaaggttctgaa |
| Ga0209741_10051251) | acttcacccaacgaggtcgcccgctggactgttaaagccgtcaccgagaagattgatggcgtaatgat |
| | tcaggtctttcgttggaaaggtgagttaatctgggcttcgcgccatggtatttggtcgaatgccgcgac |
| | cgacgcatttaaggtagcatcatcagcggtagaaaagatcttcccccgcaaaggtaattggacgctg |
| | atctgtgaattcatccacccagaccatcgtaaagccggtatgatcgattacggcgatctggtgggcctt |
| | ggagtgctttatttgcgcgacttggattctcttgaattgattcccgcacgcgagaagttcgacgacgac |
| | ctgcccagcccattgttccttcctgcgttataccccttcagccaattttgggaggcgcgcgagttcgtac |
| | agaacagtcagactcgctactttgaaggcgtcgtcttacagggtgctgaggaattaggaaatcgttta |
| | gtgaagatcaagaaccctttatatcttgacgctcttgccaccattcgcgcgatcacaccaaatcgcatt |
| | attttcatctacgaacgcctggggctgaatggggtcaaggacttatttagcctttacaaggacgttttg |
| | gatgacattccagaagcgcgtcagttggctgcggaactggaaaaggcggaagcagagttcgttgcac |
| | gttgtttggagctgcgtgagaaagaaatggaagaaatccccccggagatgcgttgggttaaaagtta |
| | tgaggtgggtagtaagaaatggaaccaaaccgtttggcgtttcgtcgcggggaaagtcagttggcag |
| | ctgaccgagccgaagcccaaaccgcccagtcgttatgagtacgacgaaatcgac 3′ |
| SEQ ID NO 6 | MEERVRVYQAIPSLERAFDIAKDAKAIAFRTSEEGLVLFNYLFSDQQLWTQVPES |
| (Protein sequence of | RNLRGIVYEQTSGRVVSLPFHKFFNPGEPASPDVSKYDFGKSLVSKKHDGYLLQTF |
| a thermostable | VYRGKVYTISRHSFKAPLVQTVLQGLWDKRHERFVLQVSEEYPQGITLLWEVIHP |
| ligase enzyme. Locus | LYPVLELPEKPSLVLLAARMTDTGDYLFPVIEGESDPPFEVKTLSVPSSFLSDGITEV |
| tag | ARWLPVSSLFENYSSWNDIKQQVKSIHRSEGYVIALFTQEGSEFVFDDFVKAKTP |
| Ga0209741_10051251) | WAFKASLLFANPGDTLVRSVVEDKVDDLVYEVLKDDPRLQAFSKAHRTLLNHIYL |
| | AYDFGLGLSQKQVEAKDAYQAAVSWSQPYGKYHPELPSVFTPLIMKAYRGASFE |
| | EVWENFKKLMENKKKLVAVSSWIELTHQLHYVEPDG |
| SEQ ID NO 7 | 5′gtctggttggtcagccgttgtgggatgttagccgtagcagcactggtaatctggttgaatggtt 3′ |
| (DNA oligonucleotide | |
| substrate) | |
| SEQ ID NO 8 | 5′atctggttggtcagccgttgtgggatgttagccgtagcagcactggtaatctggttgaatggta 3′ |
| (DNA oligonucleotide | |
| substrate) | |
| SEQ ID NO 9 | 5′ntctggttggtcagccgttgtgggatgttagccgtagcagcactggtaatctggttgaatggtn 3′ |
| (DNA oligonucleotide | |
| substrate; n = a, g, c | |
| or t at position 1 and | |
| 64) | |
| SEQ ID NO 10 | 5′gtctggttggtcagccgttgtgggatgntagccgtagcagcactggtaatctggttgaatggtg 3′ |
| (DNA oligonucleotide | |
| substrate; n = | |
| internal fluorescein- | |
| labeled thymine at | |
| position 28) | |
| SEQ ID NO 11 | 5′ctctggttggtcagccgttgtgggatgntagccgtagcagcactggtaatctggttgaatggtc 3′ |
| (DNA oligonucleotide | |
| substrate; n = | |
| internal fluorescein- | |
| labeled thymine at | |
| position 28) | |
| SEQ ID NO 12 | 5′ttctggttggtcagccgttgtgggatgttagccgtagcagcactggtaatctggttgaatggtc 3' |
| (DNA oligonucleotide | |
| substrate) | |
| SEQ ID NO 13 | 5′uaggcgucggugacaaacggccagcguuguugucucucuguucuagcuuaucgguc 3' |
| (RNA oligonucleotide | |
| substrate) | |
| SEQ ID NO 14 | 5′ctctggttggtcagccgttgtgggatgttagccgtagcagcactggtaatctggttgaatggtc 3' |
| (DNA oligonucleotide | |
| substrate) | |

TABLE 3
Pairwise sequence identity analysis of active RNA ligase 1 enzymes

| | | | | T4 | TS2126 | RM378 | A. pernix |
|---|---|---|---|---|---|---|---|
| | | | | Rnl1 | Rnl1 | Rnl1 | ligase from |
| | GBS-3074 | Ga0105160_10035846 | Ga0209741 _10051251 | ligase | ligase | ligase | US20040259123A1 |
| GBS-3074 | | 28.1% | 46.0% | 25.4% | 31.4% | 23.6% | 26.7% |
| Ga0105160_10035846 | 28.1% | | 25.5% | 22.5% | 29.5% | 22.8% | 20.6% |
| Ga0209741_10051251 | 46.0% | 25.5% | | 22.4% | 29.0% | 23.6% | 27.7% |
| T4 Rnl1 ligase | 25.4% | 22.5% | 22.4% | | 27.3% | 24.4% | 27.8% |
| TS2126 Rnl1 ligase | 31.4% | 29.5% | 29.0% | 27.3% | | 29.3% | 27.3% |
| RM378 Rnl1 ligase | 23.6% | 22.8% | 23.6% | 24.4% | 29.3% | | 21.9% |
| A. pernix ligase from | 26.7% | 20.6% | 27.7% | 27.8% | 27.3% | 21.9% | |
| US20040259123A1 | | | | | | | |
| P. furiosus ligase from | 25.4% | 25.4% | 27.7% | 25.6% | 31.5% | 19.7% | 38.4% |
| US20090061481A1 | | | | | | | |
| Thermus sp. ligase from | 27.7% | 26.7% | 23.1% | 22.4% | 30.3% | 35.7% | 24.2% |
| WO2000026381A2 | | | | | | | |
| Unspecified ligase from | 25.4% | 25.4% | 27.7% | 25.6% | 31.5% | 19.7% | 38.6% |
| WO1994002615A1 | | | | | | | |
| P. furiosus ligase from | 25.4% | 25.4% | 27.7% | 25.6% | 31.5% | 19.7% | 38.6% |
| US20110053147A1 | | | | | | | |

| | P. furiosus | Thermus sp. | Unspecified | P. furiosus |
|---|---|---|---|---|
| | ligase from | ligase from | ligase from | ligase from |
| | US20090061481A1 | WO2000026381A2 | WO1994002615A1 | US20110053147A1 |
| GBS-3074 | 25.4% | 27.7% | 25.4% | 25.4% |
| Ga0105160_10035846 | 25.4% | 26.7% | 25.4% | 25.4% |
| Ga0209741_10051251 | 27.7% | 23.1% | 27.7% | 27.7% |
| T4 Rnl1 ligase | 25.6% | 22.4% | 25.6% | 25.6% |
| TS2126 Rnl1 ligase | 31.5% | 30.3% | 31.5% | 31.5% |
| RM378 Rnl1 ligase | 19.7% | 35.7% | 19.7% | 19.7% |
| A. pernix ligase from | 38.4% | 24.2% | 38.6% | 38.6% |
| US20040259123A1 | | | | |
| P. furiosus ligase from | | 26.7% | 99.8% | 99.8% |
| US20090061481A1 | | | | |
| Thermus sp. ligase from | 26.7% | | 26.9% | 26.9% |
| WO2000026381A2 | | | | |
| Unspecified ligase from | 99.8% | 26.9% | | 100.0% |
| WO1994002615A1 | | | | |
| P. furiosus ligase from | 99.8% | 26.9% | 100.0% | |
| US20110053147A1 | | | | |

### FIGURE CAPTIONS

FIG. 1

Single-stranded DNA circularization activity of the two forms of GBS-3074 ligase in the absence or presence of different concentrations of ATP:
a) Reactions contained the adenylated form of the GBS-3074 ligase.
b) Reactions contained the unadenylated form of the GBS-3074 ligase or CircLigase ssDNA ligase.

FIG. 2

Comparable single-stranded DNA circularization efficiency of unadenylated GBS-3074 ligase in 60 minute reactions in the presence of ATP using 64 nucleotide substrates containing different terminal nucleotides.

FIG. 3

Reduced terminal nucleotide sequence bias of GBS-3074 ligase compared with CircLigase II in 45 minute single-stranded DNA circularization reactions:
a) Image of SYBR Gold-stained denaturing acrylamide gel.
b) Bands corresponding to linear oligonucleotide and circularized product were quantified and the percent circular product were determined for each condition.

FIG. 4

Rapid ligation reaction kinetics of GBS-3074 ligase compared with CircLigase II:
a) Substrate is a 64 nucleotide 5′-phosphorylated oligo with a 5′-G and 3′-G nucleotide.
b) Substrate is a 64 nucleotide 5′-phosphorylated oligo with a 5′-C and 3′-C nucleotide.

FIG. 5

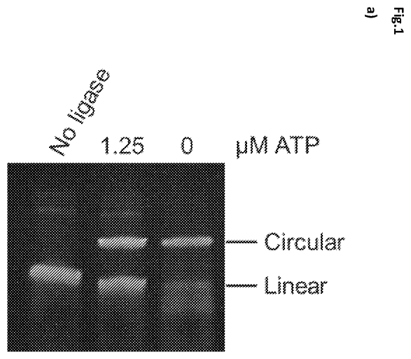

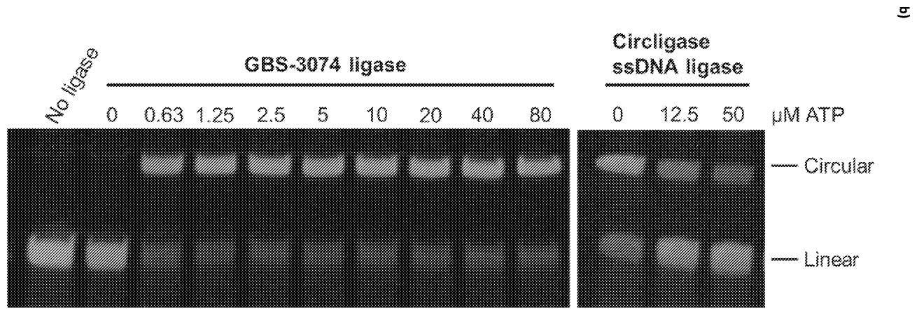

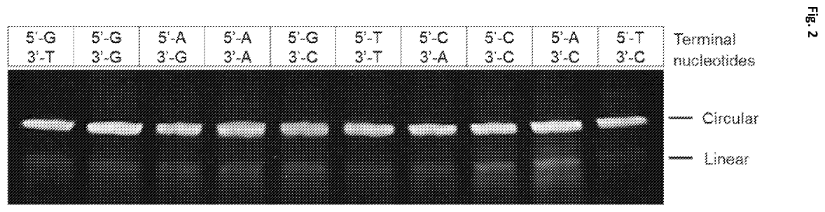

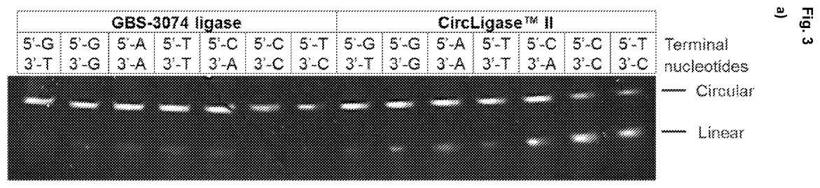

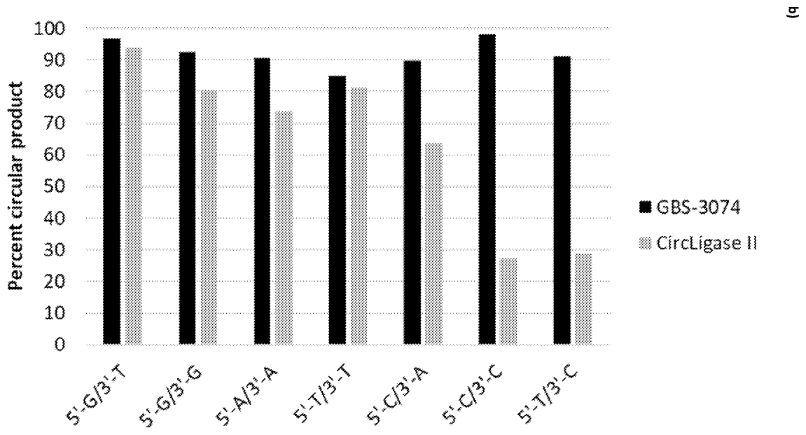

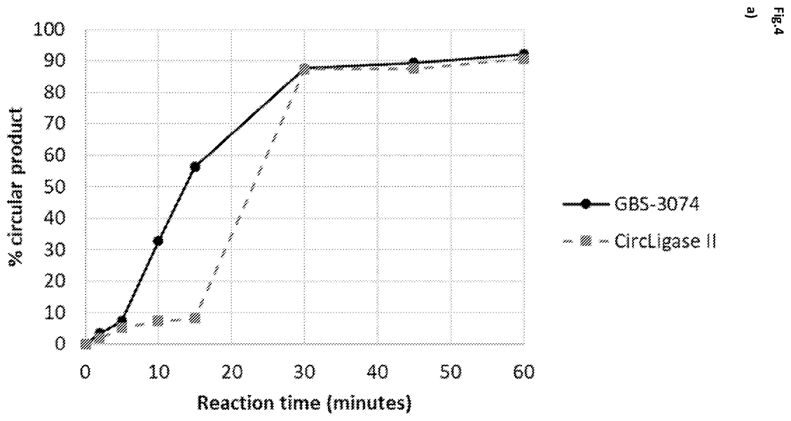

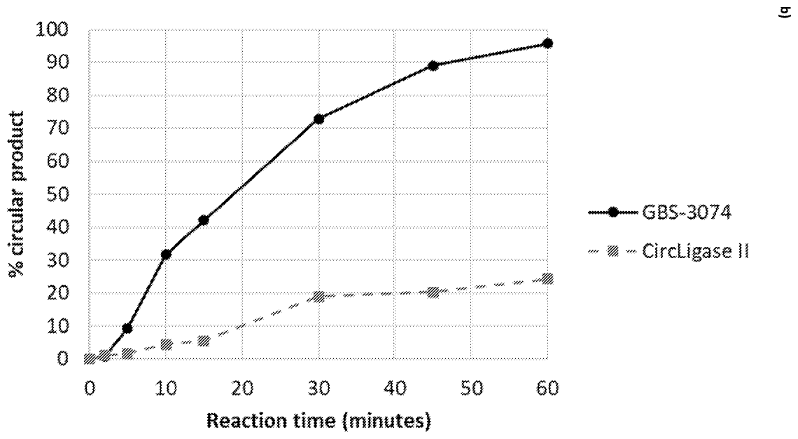

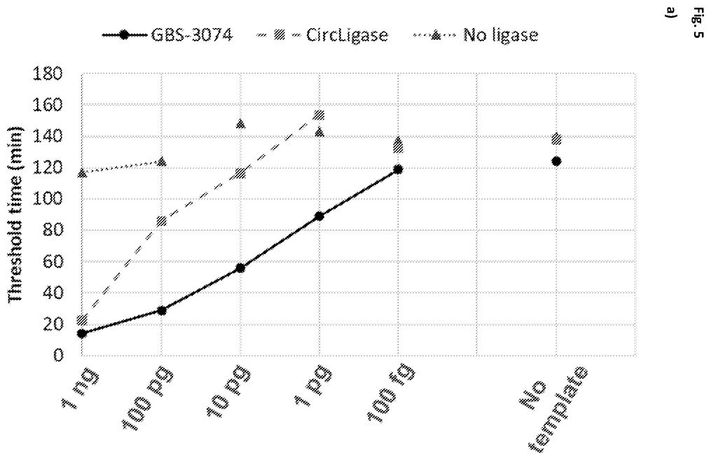

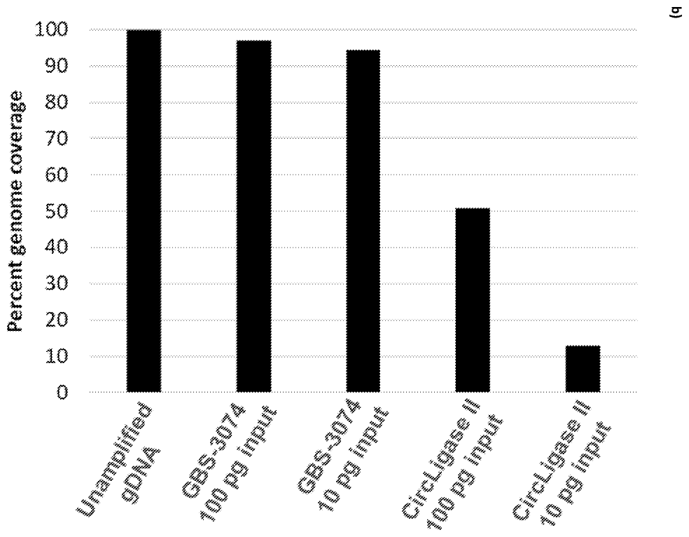

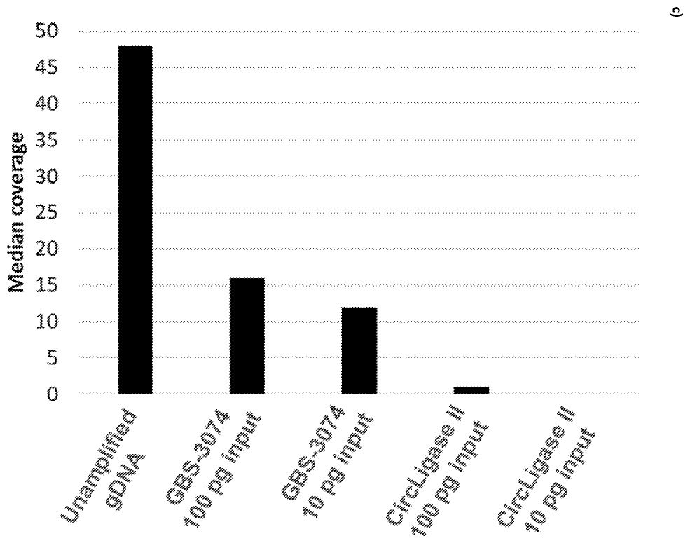

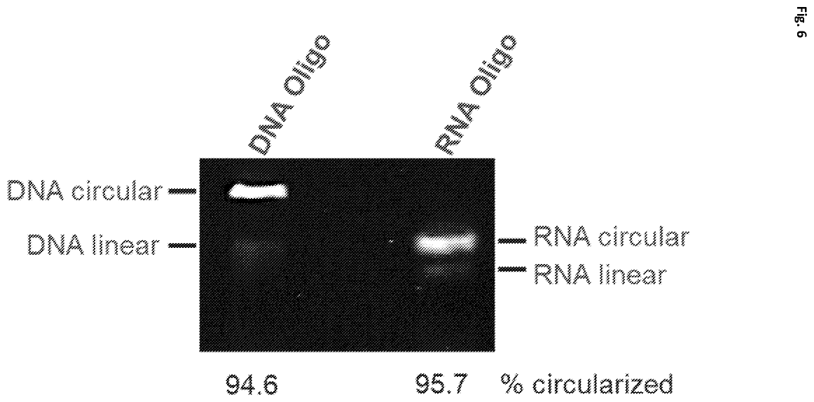

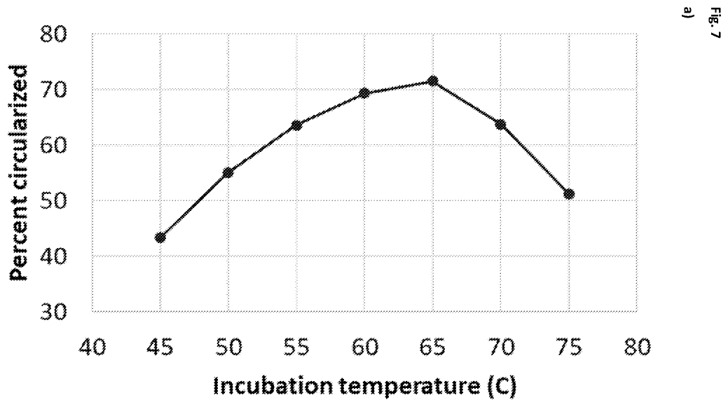

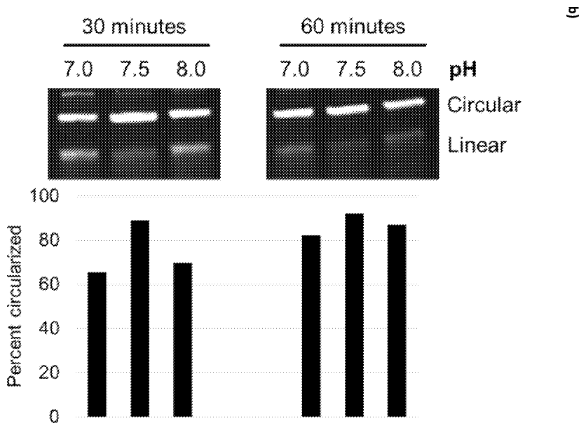

Single-stranded DNA circularization efficiency of GBS-3074 ligase using very low quantities of randomly fragmented E. coli genomic DNA substrate:
a) Rolling circle amplification kinetics of unligated sheared DNA, or circularized DNA products generated by either GBS-3074 ligase or CircLigase II using the indicated input quantities of sheared DNA.
b) Percentage of the E. coli genome mapped with Illumina MiSeq sequencing reads originating from either intact unamplified genomic DNA or the products of the sheared, low input circularized rolling circle amplification reactions.
c) Median coverage of the E. coli genome mapped with Illumina MiSeq sequencing reads originating from either intact unamplified genomic DNA or the products of the sheared, low input circularized rolling circle amplification reactions.

FIG. 6

Comparable DNA and RNA single-stranded substrate circularization efficiency using GBS-3074 ligase in 60 minute reactions in the presence of ATP using 64 nucleotide substrates.

FIG. 7

Characterization of GBS-3074 ligase optimal reaction temperature and pH
a) Graph depicting the extent of single-stranded DNA circularization at different incubation temperatures ranging from 45° C. to 75° C.
b) Image of SYBR Gold-stained denaturing acrylamide gel and graph illustrating the extent of circularization of a single-stranded DNA substrate at different reaction pH values.

1. A thermostable ligase consisting of or comprising the amino acid sequence of SEQ ID NO. 2 or a polypeptide that shares at least 60%, 65%, 70%, 75%, 80%, 85%, 90%, 95% or 98% amino acid sequence identity thereto or a derivative or fragment thereof having a ligase activity.

2. A thermostable ligase according to claim 1, wherein the ligase is capable of intramolecular ligation of an RNA or DNA molecule.

3. A thermostable ligase according to claim 1, wherein the ligase is capable of intermolecular ligation of two or more RNA and/or DNA molecules.

4. A thermostable ligase according to claims 1, wherein the RNA and/or DNA molecules are single-stranded.

5. A thermostable ligase according to claims 1, wherein the ligase does not require a bridging or splint nucleic acid molecule for ligation.

6. A thermostable ligase according to claims 1, wherein the ligase is template-independent.

7. A thermostable ligase according to claims 1, wherein the ligase is unadenylated and requires ATP for activity.

8. A thermostable ligase according to claims 1, wherein the unadenylated ligase is capable of multiple rounds of self-adenylation and catalysis in the presence of ATP.

9. A thermostable ligase according to claims 1, wherein the ligase is adenylated and inhibited in the presence of ATP.

10. A thermostable ligase according to claim 1, wherein the ligation is performed at a temperature in the range of 45° C. to 75° C., preferably in the range of 55° C. to 70° C., more preferably in the range of 60 to 65° C.

11. A nucleic acid molecule encoding a thermostable ligase according to claim 1.

12. A nucleic acid molecule consisting of or comprising the nucleic acid sequence according to SEQ ID NO. 1 or a nucleic acid sequence having at least 60%, 65%, 70%, 75%, 80%, 85%, 90%, 95% or 98% identity thereto.

13. An expression vector comprising the nucleic acid molecule of claims 11.

14. Kit comprising a thermostable ligase according to claim 1.

15. Use of a thermostable ligase according to claim 1 for rolling-circle amplification, digital nucleic acid analysis, rolling-circle transcription, isothermal amplification, amplification of low copy fragmented DNA, sequencing and next generation sequencing library preparation, whole genome sequencing, whole-genome bisulfate sequencing, amplification of cDNA ends, 3′ end labeling of RNA, oligonucleotide synthesis, cDNA adapter ligation, rapid amplification of cDNA ends, ligation of single-stranded primer products for PCR.

16. An expression vector comprising the nucleic acid molecule of claim 12.

17. Kit comprising an expression vector according to claim 13.

18. Kit comprising an expression vector according to claim 16.


SEQUENCE LISTING (14 sequences)

SEQ ID NO: 1 - DNA, 1155 bp, Unknown
  DNA sequence of a thermostable ligase enzyme referred to as GBS-3074 ligase. Locus tag Ga0072500_1423074
atgacgttatatgagttacgtaagggcttagaagcggtgaaacattacatccttaacaacgacgcatcagcgtcggggtataacgacgacttactggagcgtatggtatgggtgaatcacgaatatgatctggtcctgttaaactacaaagatgccactgctatgatcctgcataacgaagggttacagtggacaccttttttgcgtgtttgtcgtggggttgtatttacgccttcaggcgaactggtctcgttgcccttacacaaattcttcaatgtaaaggagaacgaagagacctcgttggctaatatcgctaattggcctctgcgtagtgctaccgagaaggtagacggggtcatgattcaggtgttcttccacccactgcgcaaggaaattacctatgccagtcgctggcgcatttggtccgacgcagccatcactgcgtttaaattagcgaactcagcgctgactaacgcggtaatcccaaagctgaatgcctctttcggtgaaggaaagtggacgttaatttgtgaattaatccatccagagcatcgtcagcccggaatggttagctatggggacttacaagcgttggtgcttctgtacgtccgcaaattagacgatcttgagttgattccagccgtagaattgtttaaggataacgaattgccgcccccacttatgctgccacagcaatatttaattgtgtccgcactggaagcccttgagaaagtcaagcaggccaagcacgcgaattgggagggtattgttgttcagggggcaatggagggtgggaatcgtctggtgaagatgaaaaaccctctgtacttagagggagtaaacgccgtgaaaaacttgaatcgcattttaaagatctatgaagcacaggggcgcgaaggcgtggaaaacctgttcctgctgtacgcatcctacttggacgatgtcccgcacatcgtgggtcttcgcgatttgttgtacaagaccgaggacgagattaacaactacgctaagcagttgcgcgaatcgacacaggacgtgactaccttgcctcgtgaatggcgttgggtcaaatcttatgacgtcggcaatgacaaatggcagcgctgcgtccgccgtatggtactgcaaaaagtgaacgcaggcggtcgtaagtaa

SEQ ID NO: 2 - protein, 384 aa, Unknown
  Protein sequence of a thermostable ligase enzyme referred to as GBS-3074 ligase. Locus tag Ga0072500_1423074
MTLYELRKGLEAVKHYILNNDASASGYNDDLLERMVWVNHEYDLVLLNYKDATAMILHNEGLQWTPFLRVCRGVVFTPSGELVSLPLHKFFNVKENEETSLANIANWPLRSATEKVDGVMIQVFFHPLRKEITYASRWRIWSDAAITAFKLANSALTNAVIPKLNASFGEGKWTLICELIHPEHRQPGMVSYGDLQALVLLYVRKLDDLELIPAVELFKDNELPPPLMLPQQYLIVSALEALEKVKQAKHANWEGIVVQGAMEGGNRLVKMKNPLYLEGVNAVKNLNRILKIYEAQGREGVENLFLLYASYLDDVPHIVGLRDLLYKTEDEINNYAKQLRESTQDVTTLPREWRWVKSYDVGNDKWQRCVRRMVLQKVNAGGRK

SEQ ID NO: 3 - DNA, 1272 bp, Unknown
  DNA sequence of a thermostable ligase enzyme. Locus tag Ga0105160_10035846
atggaagagcgggtccgtgtttatcaagctataccatccctggaacgcgcgtttgacatagctaaagacgctaaggccatagcgtttcgtacctcggaagaaggacttgtattatttaactatttgttttctgaccaacagctgtggacacaggtacccgagtcgcgtaacttgagaggtattgtctatgagcaaacgtctggacgggtggtctctctgcccttccataagttttttaacccgggggagccagcttctccggacgtttcaaaatacgattttggaaagtcacttgtctccaaaaagcacgatggatacttgctgcagacgtttgtgtaccgtgggaaagtctacactatctccagacactcttttaaggctccactggttcaaacagtcttacagggcttatgggacaagcgccacgaacgttttgtattacaggtctctgaggagtatccgcagggaattacactgttgtgggaagttatacatcccctttatccagtcctggaacttccagaaaagccttcactggtcttattagctgctcgcatgaccgacacaggggattacttattccccgtaatagagggagaatctgacccaccgtttgaggttaaaacgctgtcagtacctagtagtttcttatcagatggaatcacggaggtagcccgttggcttcccgtgtctagtcttttcgaaaattactcgtcctggaacgacataaaacagcaggtcaagtcaattcaccgctctgaaggatatgttatagcgttgtttacacaagaggggtccgaattcgtttttgacgatttcgtaaaagctaagactccttgggcgttcaaagcgtccttgttattcgctaaccctggggatacgcttgtgcgttcagtggtcgaggataaggtggatgatcttgtgtacgaggtgttaaaagatgatccgcgtctgcaagcatttagtaaggcccacagaacgttgttaaaccacatctatcttgcctacgatttcggtcttggtctgagtcaaaagcaggtcgaagcgaaagatgcttaccaggccgccgtgtcatggtcacagccctacggtaaataccacccagagttgccgagcgtatttaccccgttgataatgaaggcctaccgcggtgcgtcctttgaagaagtttgggagaatttcaaaaaattaatggagaacaaaaaaaagttggttgcagtttctagctggattgaacttacccatcaacttcactacgtggagccagatgga

SEQ ID NO: 4 - protein, 424 aa, Unknown
  Protein sequence of a thermostable ligase enzyme. Locus tag Ga0105160_10035846
MEERVRVYQAIPSLERAFDIAKDAKAIAFRTSEEGLVLFNYLFSDQQLWTQVPESRNLRGIVYEQTSGRVVSLPFHKFFNPGEPASPDVSKYDFGKSLVSKKHDGYLLQTFVYRGKVYTISRHSFKAPLVQTVLQGLWDKRHERFVLQVSEEYPQGITLLWEVIHPLYPVLELPEKPSLVLLAARMTDTGDYLFPVIEGESDPPFEVKTLSVPSSFLSDGITEVARWLPVSSLFENYSSWNDIKQQVKSIHRSEGYVIALFTQEGSEFVFDDFVKAKTPWAFKASLLFANPGDTLVRSVVEDKVDDLVYEVLKDDPRLQAFSKAHRTLLNHIYLAYDFGLGLSQKQVEAKDAYQAAVSWSQPYGKYHPELPSVFTPLIMKAYRGASFEEVWENFKKLMENKKKLVAVSSWIELTHQLHYVEPDG

SEQ ID NO: 5 - DNA, 1146 bp, Unknown
  DNA sequence of a thermostable ligase enzyme. Locus tag Ga0209741_10051251
atgactattcagcaactgcgcgatggattagcccaagtaatggagttcgtgcgcaagaaccagtacccttcggaaatcggtcgttattttatccgccgtcgctgggaaaatttagtcctgttaaactacgctgactcagccgtttacaaattctcggcagacgagtggacgccgccgatgcgtgtttgtcgcggagtgatcgtaacggatgacggatcacaagtggtcagctttcctttccataaattttttaatgttggggaaggttctgaaacttcacccaacgaggtcgcccgctggactgttaaagccgtcaccgagaagattgatggcgtaatgattcaggtctttcgttggaaaggtgagttaatctgggcttcgcgccatggtatttggtcgaatgccgcgaccgacgcatttaaggtagcatcatcagcggtagaaaagatcttcccccgcaaaggtaattggacgctgatctgtgaattcatccacccagaccatcgtaaagccggtatgatcgattacggcgatctggtgggccttggagtgctttatttgcgcgacttggattctcttgaattgattcccgcacgcgagaagttcgacgacgacctgcccagcccattgttccttcctgcgttataccccttcagccaattttgggaggcgcgcgagttcgtacagaacagtcagactcgctactttgaaggcgtcgtcttacagggtgctgaggaattaggaaatcgtttagtgaagatcaagaaccctttatatcttgacgctcttgccaccattcgcgcgatcacaccaaatcgcattattttcatctacgaacgcctggggctgaatggggtcaaggacttatttagcctttacaaggacgttttggatgacattccagaagcgcgtcagttggctgcggaactggaaaaggcggaagcagagttcgttgcacgttgtttggagctgcgtgagaaagaaatggaagaaatccccccggagatgcgttgggttaaaagttatgaggtgggtagtaagaaatggaaccaaaccgtttggcgtttcgtcgcggggaaagtcagttggcagctgaccgagccgaagcccaaaccgcccagtcgttatgagtacgacgaaatcgac

SEQ ID NO: 6 - protein, 424 aa, Unknown; identical to SEQ ID NO: 4
  Protein sequence of a thermostable ligase enzyme. Locus tag Ga0209741_10051251

SEQ ID NO: 7 - DNA, 64 bp, Unknown
  DNA oligonucleotide substrate
gtctggttggtcagccgttgtgggatgttagccgtagcagcactggtaatctggttgaatggtt

SEQ ID NO: 8 - DNA, 64 bp, Unknown
  DNA oligonucleotide substrate
atctggttggtcagccgttgtgggatgttagccgtagcagcactggtaatctggttgaatggta

SEQ ID NO: 9 - DNA, 64 bp, Unknown
  allele 1;64: DNA oligonucleotide substrate; n = a,g,c or t at position 1 and 64
ntctggttggtcagccgttgtgggatgttagccgtagcagcactggtaatctggttgaatggtn

SEQ ID NO: 10 - DNA, 64 bp, Unknown
  modified_base 28: DNA oligonucleotide substrate; n = internal fluorescein-labeled thymine at position 28
gtctggttggtcagccgttgtgggatgntagccgtagcagcactggtaatctggttgaatggtg

SEQ ID NO: 11 - DNA, 64 bp, Unknown
  modified_base 28: DNA oligonucleotide substrate; n = internal fluorescein-labeled thymine at position 28
ctctggttggtcagccgttgtgggatgntagccgtagcagcactggtaatctggttgaatggtc

SEQ ID NO: 12 - DNA, 64 bp, Unknown
  DNA oligonucleotide substrate
ttctggttggtcagccgttgtgggatgttagccgtagcagcactggtaatctggttgaatggtc

SEQ ID NO: 13 - RNA, 56 nt, Unknown
  RNA oligonucleotide substrate
uaggcgucggugacaaacggccagcguuguugucucucuguucuagcuuaucgguc

SEQ ID NO: 14 - DNA, 64 bp, Unknown
  DNA oligonucleotide substrate
ctctggttggtcagccgttgtgggatgttagccgtagcagcactggtaatctggttgaatggtc